(19) United States
(12) Patent Application Publication
(10) Pub. No.: US 2006/0074032 A1
(43) Pub. Date: Apr. 6, 2006

(54) Synthesis and separation of optically active isomers of erythromycin and their biological actions

(75) Inventors: John Somberg, Lake Forest, IL (US); Vasant Ranade, Lake Forest, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Academic Pharmaceuticals, Inc.

(21) Appl. No.: 11/254,010

(22) Filed: Oct. 19, 2005

Related U.S. Application Data
(63) Continuation of application No. 10/432,913, filed on Nov. 17, 2003, now abandoned.
(60) Provisional application No. 60/250,292, filed on Nov. 29, 2000.

(51) Int. Cl. A61K 31/7048; C07H 17/08

(52) U.S. Cl. 514/29; 536/7.2

(57) ABSTRACT

The present invention provides methods for purifying and using optically active isomers of erythromycin as well as compositions comprising such optically active isomers. Such optically active isomers having desired actions as an antibiotic substantially separable from undesirable effects on GI motility and the cardiac potassium channels such that the cardiac action potential is not prolonged and the QT interval on the surface EKG (electrocardiogram) is not increased, such that the erythromycin can be useful for more effective therapy of systemic infections. Also disclosed are methods for assaying the levels of such isomers present in the biological fluids.

This application claims the benefit of U.S. Provisional Application Ser. No. 60/250,292, filed Nov. 29, 2000, which is incorporated herein by reference in its entirety.

### FIELD OF THE INVENTION

The present invention relates generally to the resolving of enantiomers of erythromycin. More particularly, the invention relates to the identification of the biological activity of the different enantiomers of erythromycin.

### BACKGROUND OF TES INVENTION

Erythromycin is an orally effective antibiotic discovered in 1952 by McGuire and colleagues from the metabolic products of a strain of Streptomyces erythreus, originally obtained from a soil sample collected in the Philippine archipelago (McCormick M H, et al. Vancomycin, a new antibiotic I. Chemical and biologic properties. Antibiotics Annual, 1955-1956. Medical Encyclopedia, Inc. New York, 1956, pp. 606-611). Erythromycin is one of the macrolide antibiotics so named because they contain a multi-constituent lactone ring, to which is attached one or more deoxy sugars. While newer macrolides are now available with improved acid stability, tissue penetration and a broader spectrum of anti-microbial activity, erythromycin is still often prescribed, possessing a unique antibacterial activity. Erythromycin is bacteriostatic by inhibiting protein synthesis, binding reversibility to the 50 S ribosomal subunits of sensitive microorganisms (Brisson-Noël, A, et al., Mechanism of action of spiramycin and other macrolides. J. Antimicrob Chemother., 22 suppl. B:13-23, 1988).

However, erythromycin can have life threatening side effects, such as QT prolongation (Tschida S J, et al., QTc-interval prolongation associated with slow intravenous erythromycin lactobionate infusions in critically ill patients: a prospective evaluation and review of the literature. Pharmacotherapy 1996 July-August; 6 (4):663-74; Antzelevitch C, et al. Cellular and ionic mechanisms underlying erythromycin-induced long QT intervals and torsade de pointes. J Am Coll Cardiol 1996 December; 28 (27):1836-48). QT prolongation is a problem especially in patients on other medications that prolong the QT interval (Laine K, et al. Frequency and clinical outcome of potentially harmful drug metabolic interactions in patients hospitalized on internal and pulmonary medicine wards: focus on warfarin and cisapride. Ther Drug Monit 2000 October; 22 (5):503-9) or female patients that tend to have a longer QT interval initially (Drici M D, et al. Cardiac actions of erythromycin: influence of female sex. JAMA 1998 Nov. 25; 280 (20):1774-6). In addition, QT prolongation in these patients places them at risk for ventricular tachycardia, a possible fatal cardiac rhythm of the Torsade de pointe variety (Ebert S N, et al. Female gender as a risk factor for drug induced cardiac arrhythmias: evaluation of clinical and experimental evidence. J. Womens Health 1998 June; 7 (5):547-57).

The QT prolonging action of many agents is believed to be due to inhibition of the potassium rectifying current (Daleau P et al. Erythromycin blocks the rapid component of the delayed rectifier potassium current and lengthens repolarization of guinea pig ventricular myocytes. Circulation 1995 Jun. 15; 91 (12):3010-6). Erythromycin is known to inhibit the potassium rectifying current IKᵣ (Drici M D, Barhanin J. Cardiac K+ channels and drug-acquired long QT syndrome. Therapie 2000 January-February; 55 (1):185-93). A number of other agents that inhibit I_{Kr} are also known to increase GI motility causing diarrhea: quinidine, cissapride and nortriptiline.

While erythromycin is frequently prescribed, its use is constrained by severe GI distress and diarrhea, which are often limiting side effects (55^{th} Ed. 2001; Page 447, Physicians Desk Reference PDR; Otterson M F, Sarna S K. Gastrointestinal motor effects of erythromycin. Am J Physiol 1990 September; 259 (3 Pt 1):G355-63). prolongation, especially in female patients taking other medications that can prolong the QT interval. Abbott Pharmaceuticals has developed a film coated erythromycin tablet to decrease GI irritability and diarrhea that is very expensive and only offers a minimal reduction in the GI side effects.

Other agents cause QT prolongation and diarrhea through an increased GI propulsive activity. Cissapride causes QT prolongation and is a prokinetic GI motility agent (Desta Z, et al. Stereoselective determination of cisapride, a prokinetic agent, in human plasma by chiral high-performance liquid chromatography with ultraviolet detection: application to pharmacokinetic study. J. Chromatogr B Biomed Sci Appl 2000 Jul. 21; 744 (2):263-72; Gray VS. Syncopal episodes associated with cisapride and concurrent drugs. Ann Pharmacother 1998 June; 32(6); 648-51). Quinidine, an antiarrhythmic drug causes QT prolongation, Torsade de pointe arrhythmias and propulsive diarrhea which often complicate its use (Vaughn Williams E M. Class I Anti-arrhythmic actions, chapter 2. In Control of Cardiac Rhythm by Vaughn Williams E M, Somberg J C, 1998, Lippincott-Raven Publishers, New York; Van Nueten J M, et al. Inhibition of dopamine receptors in the stomach: an explanation of the gastrokinetic properties of domperidone. Life Sciences 1978; 23:453-458). Recently, we have shown that the toxic side effects of quinidine are the result of stereoisomers of quinidine. One stereoisomer blocks the K⁺ ion channel, IKᵣ responsible for QT prolongation and possibly enhanced GI contractility. The primary antiarrhythmic action of quinidine on the sodium channel was unaffected. In an analogous fashion we believe that the toxic side effects of erythromycin may be separable from the beneficial anti-bacterial action of erythromycin. Since the toxicity profile of erythromycin is known (carcinogenicity, teratotoxicity, etc), the successful isolation of an optimal erythromycin stereoisomer would be subject to immediate clinical testing.

Erythromycin exists of a number of forms called A, B, C, D, E and F (see Formula 1 and Table 1, below). Erythromycin consists of the aglycone erythromolide A; the aminopryas, desosamine and the neutral soyas, cladinose. This compound is a white crystalline powder, practically odorless and has a bitter taste. Erythromycin and these related compounds have been designated as A, B, C, D, E and F, which differ from each other with respect to the fact that they have different substituents attached to the erythromycin molecule. These compounds possess the inherent chirality of the erythromycin molecule. The following discussion of chiral separation will focus on erythromycin A, but the techniques are generally applicable to other racemic mixtures of like molecules, including the erythromycin epimers listed in Table 1, below.

There are ten chiral centers in the erythromycin molecule, yielding a multitude of possible sterioisomers. Optically active sites are at carbons 2, 3, 4, 5, 6, 8, 10, 11, 12 and 13 positions of the macrolide ring (See Formula 1, below). Each of these isomers can have different actions, because they have different shapes facilitating or preventing binding to receptor/binding sites or interacting with other enzymes or pharmaceutical agents. The optically active isomers can be separated, purified and characterized using the technique of chiral high pressure liquid chromatography (HPLC) that is described below. The expectation of differential effects of enantiomers of erythromycin is supported by the many known examples of different enantiomeric compounds having significantly different biological activity. For example, the different enantiomers of beta-blockers (e.g. levalbuterol and beta-amino alcohols), amphetamine (AP), methamphetamine (MAP), and penicillamine have different pharmacological activities and pharmacokinetic behaviors. The S-isomers of AP and MAP are each approximately five times more active on the central nervous system (CNS) than their respective R-isomer.

TABLE 1

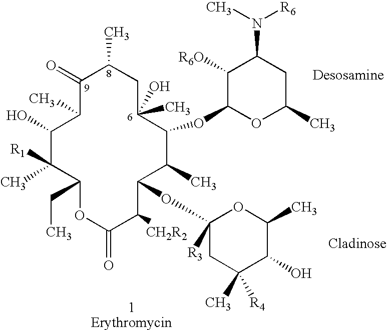

| | Erythromycin Epimers | | | | | |
|---|---|---|---|---|---|---|
| | R1 | R2 | R3 | R4 | R5 | R6 |
| Erythro- | OH | H | H | OCH₃ | CH₃ | H |
| mycin A | | | | | | |
| Erythro- | H | H | H | OCH₃ | CH₃ | H |
| mycin B | | | | | | |
| Erythro- | OH | H | H | OH | CH₃ | H |
| mycin C | | | | | | |
| Erythro- | H | H | H | OH | CH₃ | H |
| mycin D | | | | | | |
| Erythro- | OH | —O— | | OCH₃ | CH₃ | H |
| mycin E | | | | | | |
| Erythro- | OH | OH | H | OCH₃ | CH₃ | H |
| mycin F | | | | | | |
| N-demeth- | OH | H | H | OCH₃ | H | H |
| ylerythro- | | | | | | |
| mycin A | | | | | | |
| Erythro- | OH | H | H | OCH₃ | CH₃ | C₃H₃COOC(CH₂)₂CO |
| mycin | | | | | | |
| Ethyl- | | | | | | |
| succinate | | | | | | |

The commercial success of enantiomers with specific biological activities is demonstrated by the antihistamine terfenadine, the psychoactive agent fluoxetine and the prokinetic gastrointestinal agent cisapride. Terfenadine was originally sold as a racemate mixture of R- and S-isomers under the name Seldane®. After discovering that racemic terfenadine was preferentially oxidized in rats to form a carboxylic acid metabolite enriched in the R-enantiomer, Hoechst Marion Roussel began marketing the R-isomer of terfenadine as Allegra® (fexofenadine). A single isomer preparation of fluoxetine (Prozac) is under development and a single isomer version of Zyrtec (cetirizine) may be available in the near future. A single isomer version of cisapride is being developed, norcisapride, which has a different receptor binding profile than the parent racemic drug.

Preliminary data regarding the pharmacodynamics of enantiomers, such as that mentioned above, suggest that individual isomers can possess significant differences in receptor-binding profiles and follow different courses of absorption, distribution, metabolism and excretion. As such, the administration of single isomers may significantly reduce if not elimate drug interactions mediated by the effect of enantiomers on different biological receptors. Similar to other racemic compounds, it is expected that individual enantiomers of erythromycin are responsible for the diversity of effects displayed by such compounds (e.g., action on cardiac potassium channels versus effects on GI motility). The ability to identify isomers of erythromycin with differential effects on cardiac potassium channels and GI motility would offer considerable potential clinical benefits. For example, specific enantiomers of erythromycin could be used as drugs for blocking only cardiac potassium channels while not causing diarrhea. Alternatively, an enantiomer of erythromycin not effecting the cardiac potassium channels, but increasing GI motility or mucosal secretion could be used as novel treatment for constipation or gastroesophageal reflux disease (GERD). Therefore, the isolation of specific enantiomers of erythromycin could lead to a safer, less toxic and less pro-arrhythmic compounds than racemic erythromycin.

As noted above, the erythromycin molecule has ten possible chiral centers. Some, but not others, of the chiral configurations of erythromycin may be primarily responsible for the GI and cardiac effects. The isomers can be separated using HPLC and chiral columns, ideally separating enantiomers primarily mediating the adverse effects from chiral isomers responsible for the active antibiotic effects. Such separated chiral isomers could then reduce the potential toxicity of erythromycin, making its use safer and better tolerated and thus increasing its use and its effectiveness clinically.

### SUMMARY OF THE INVENTION

The present invention provides methods for purifying, identifying and using optically active isomers of erythromycin as well as compositions comprising such optically active isomers. Such optically active isomers having desired actions on cardiac potassium channel function substantially separable from undesirable effects on GI motility can be useful for more effective antibiotic therapy. Also disclosed are methods for assaying the levels of such isomers present in the biological fluids.

In general, the present invention relates to optically active isomers of erythromycin and to methods of synthesis, isolation, purification, and systems using the same. The invention also relates to the use of optically active isomers of erythromycin to specifically block cardiac potassium channels, as well as treating constipation or gastroesophageal reflux disease (GERD) by increasing gastrointestinal (GI) motility or by increasing luminal secretion or blocking luminal fluid re-absorption. In one embodiment, the present invention provides methods of assaying the presence of optically active isomers of erythromycin in biological fluids. In another embodiment, the invention provides for assays for measuring the effects of enantiomers of erythromycin on cardiac potassium channels, as well as contractility and secretory assays for determining GI motility activity.

### BRIEF DESCRIPTION OF THE DRAWINGS

The following detailed description, given by way of example, but not to limit the invention solely to the specific embodiments described, may best be understood in conjunction with the accompanying drawings, in which:

FIG. 1 shows the results of HPLC, showing a smaller Peak 1 having a noticeable shoulder (asterisk) and a larger Peak 2. Chiral separation of erythromycin was performed using Spectra-Physics BPLC instrument and UV variable wavelength detector set at 288 nm. For chiral separation, the chromatographic columns were two pre-packed 25 mm×4.6 m ID cyclobond 1 Altech Associates (5 μm particle size) attached together. The mobile phase consisted of CH₃CN: MeOH: 0.2 m ammonium acetate: H₂O (45:10:10:25 v/v) and the flow rate was 0.8 ml/min. A quantity of 10 μL solution of erythromycin having a concentration of 10 mg/ml in methanol was injected onto the column. The chromatogram indicated at least two major peaks, which were designated as Peak 1 and Peak 2. Peak 1 had a retention time of 6.2 minutes and Peak 2 had retention time of 7.3 minutes.

FIG. 1B is a tracing of a HPLC output using different pH conditions in which Peak 1 apparently lacks the shoulder and other small peak appears at longer retention times.

FIG. 2 shows the results of mass spectrometry analysis of Peak 1 and Peak 2 of FIG. 1. Eluents corresponding to these two peaks were collected separately and analyzed by mass spectrometry. Peak 1 and Peak 2 indicated presence of erythromycin. The mass spectra of Peaks 1 and 2 were compared to that of the reference standard of erythromycin. These results show that Peaks 1 (FIG. 2B) and 2 (FIG. 2C) contain isomeric erythromycins compared to an erythromycin A standard (FIG. 2A).

FIG. 3 shows the results of studies of the effects of erythromycin on gastric motility as measured in the rat ileum preparation.

FIG. 4 is a graphical representation of the reduction of current through a potassium channel (I_{HERC}, normalized) produced by various concentrations of erythromycin (filled triangles, IC₅₀=2.4×10⁻⁵ M), peak 1 (open circles, IC₅₀=2.3 M) and peak 2 (filled circles, IC₅₀=2.2×10⁻⁵ M). Each data point is the average of three measurements; error bars represent ± one standard deviation.

### DETAILED DESCRIPTION OF THE INVENTION

The present invention relates to the isolation of enantiomers of erythromycin with the following biological functions: 1) action on cardiac potassium channels and increasing GI motility; 2) action on motilin receptors thus increasing GI motility; 3) action on cardiac potassium channels, without increasing GI motility; 4) increasing GI motility, without action on cardiac potassium channels; 5) antibiotic action without effect on potassium channels and no effect on GI motility; 6) antibiotic action without effect on cardiac potassium channels but still augmenting GI contractility; and 7) antibiotic action without an effect on GI contractility but still acting on the cardiac potassium channels. This set of desirable characteristics is illustrated in Table 2 below.

TABLE 2

| | | Cardiac Potassium Channels | |
|---|---|---|---|
| | | Action | No Action |
| GI | Action | 1 | 4, 6Ab |
| Motility | Action on Motilin Receptors | | 2 |
| | No action | 3, 7Ab | 5Ab |

5Ab, 6Ab, 7Ab having antibiotic action

In addition, the present invention relates to methods of isolating enantiomers of erythromycin using chiral columns in combination with high pressure liquid chromatography. Chiral columns have been used to effectively separate stereoisomers (Chan, K. Y., et al., (1991). Direct enantiomeric separation of terfenadine and its major acid metabolite by high-performance liquid chromatography, and the lack of stereoselective terfenadine enantiomer biotransformation in man, J. Chromatogr., 571 (1-2):291-7). In addition, chiral columns can be used for determining enantiomeric purity.

Generally, resolution of enantiomers of erythromycin can be optimized through a combination of altering the composition of the mobile phase and altering the specific packing materials of the chiral columns. Separations are performed using non-polar organic phases (e.g. heptane, iso-octane, etc.) with polar organic additives, such as tetrahydrofuran, alcohols, chlorinated hydrocarbons or similar solvents with or without buffer such as phosphate or borate. Often, the addition of a small amount of a strong acid (e.g. trifluoroacetic acid) to the mobile phase will considerably improve separation of the isomers. Anion exchange chromatography with aqueous buffers using salt or pH gradients can also be used to effectively resolve enantiomers of erythromycin.

The present invention also relates to isolating isomers of erythromycin after selective epimerization of protons adjacent to the aromatic ring. Epimerization is usually accomplished by refluxing a solution of the isomers in an acidic medium. However, epimerization is not limited to this category of chemical reaction. Due to ten chiral centers, erythromycin independently can potentially have a total of approximately 1000 enantiomers, each of which can possess distinct pharmacological properties. However, some of the enantiomers of erythromycin may not readily occur due to the steric hindrance of the rigid bicyclo ring system that does not allow conformational flexibility. If any of the enantiomers of erythromycin are not present in appreciable amounts in the preparations undergoing separation, they can be synthesized.

The present invention also relates to assays to determine the electrophysiological activity of erythromycin compounds by using electrical measurements, such as voltage clamp techniques, patch clamp techniques and as well as other single and multiple electrode techniques. In addition the present invention relates to assays to determine the biological activity of erythromycin compounds by performing GI motility studies.

### EXAMPLES

### Example 1

### Chromatographic Isolation of Erythromycin Isomers

Typical Chromatographic Method for Isolation of Erythromycin Isomers

The identification of each isomer of erythromycin can be made using a combination of 2- or 3-dimensional high resolution NMR (¹³C and proton) spectroscopy using a chiral shift reagent, mass spectrometry, and optical activity. In order to obtain isomers of erythromycin having the desired optical purity, eluted samples can be re-chromatographed.

Analysis of the isomers present in the peaks in the chromatograms and their chiral excess purity can be determined in each case by high resolution NMR spectroscopy using a chiral shift reagent Based on this information, and the determination of molecular weight by mass spectrometry and assay of optical activity (ORD), structural configurations can be assigned to each isomer. Reference standards that are characterized and are optically pure are compared to the isolated isomers that are obtained after their chromatographic separation for confirmation of purity and identity.

HPLC Instrumentation

The chromatographic system consisted of a Waters Model 510 pump supplied with a high-sensitivity noise filter (Waters Lot No. 25200), a WISP 710B automatic injector and a Nova-Pak C₁₈ column (Waters Assoc., Mississauga, Canada). The column effluent was monitored by a Waters M460 electrochemical detector in the oxidative mode with the amperometric cell potential set at +0.9 V (vs. Ag/AgCl reference electrode). The detector was interfaced with a HP 3390 data system (Hewlett-Packard Canada, Montreal, Canada). The mobile phase was pumped at a flow-rate of 1.1 ml/min (53 bar).

Chiral separation is performed using a Spectra-Physics HPLC instrument and UV variable wavelength detector set at 254 nm. The chromatographic column is a pre-packed 25 mm×4.6 mm ID Cyclobond I (5 μm) operated with a methanol—0.014 M sodium perchlorate (75:25 v/v) mobile phase, at a flow rate of 0.2 ml/min. Alternatively, a pre-packed 150 mm×4 mm ID Resolvosil BSA-7 column (5 μm) may be operated isocratically with 0.05 M sodium phosphate buffer (pH 3.0)—acetonitrile (73:27 v/v) at a flow rate of 0.2 ml/min.

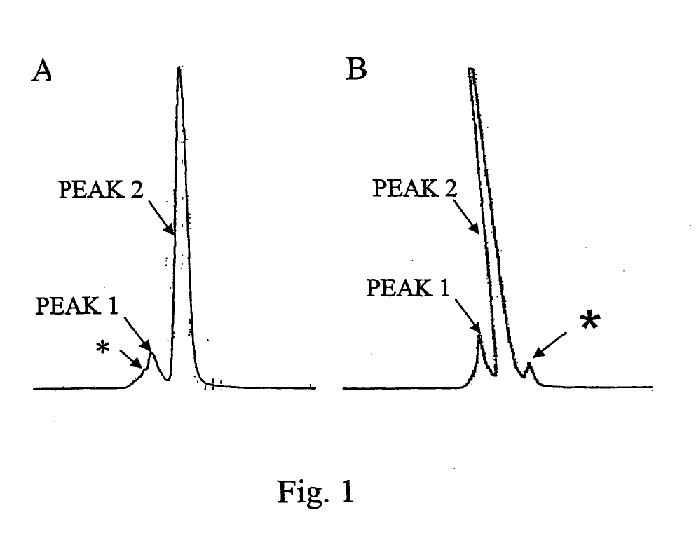

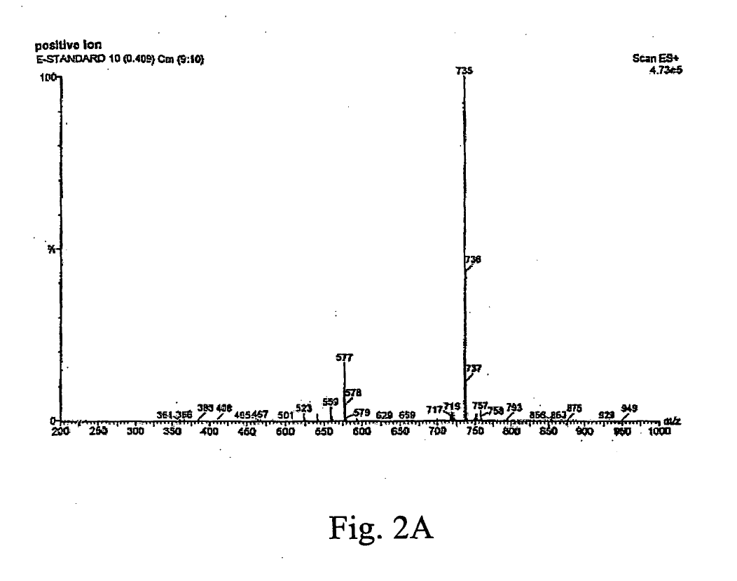

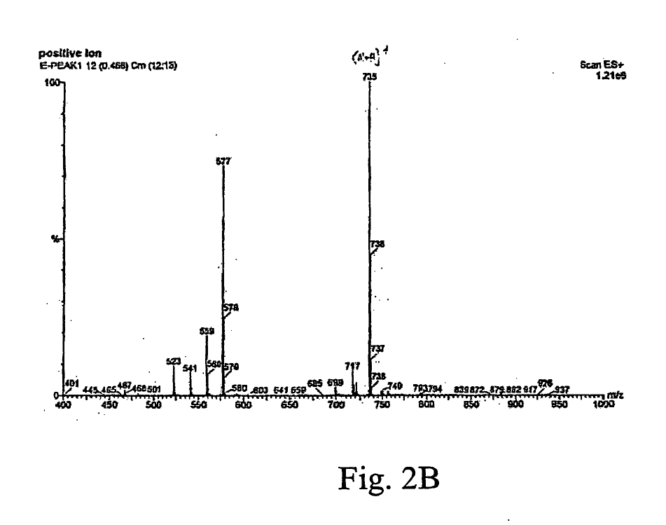

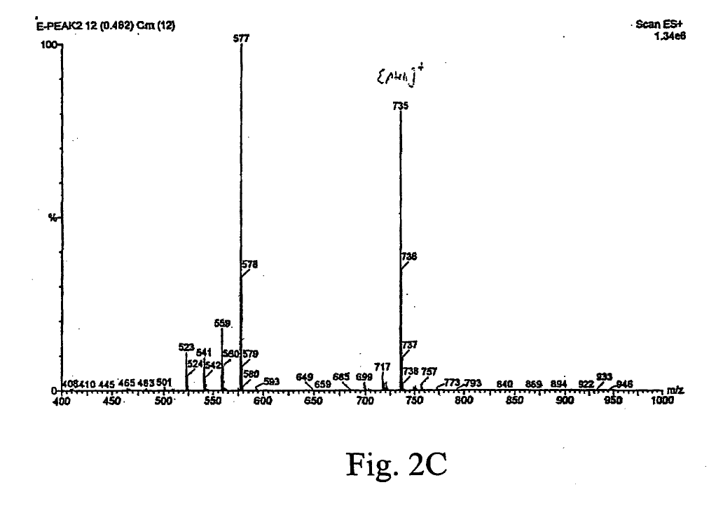

A quantity of 10 μl solution of erythromycin having a concentration of 10 mg/ml in methanol was injected onto the column. The chromatogram indicated at least two major peaks, which were designated as peak 1 and peak 2. Peak 1 had retention time of 6.2 minutes and peak 2 had retention time of 7.3 minutes (FIG. 1A). Eluents corresponding to these two peaks were collected separately and analyzed by mass spectrometry (FIG. 2). Peak 1 and peak 2 indicated presence of erythromycin. Their mass spectra (FIGS. 2B and 2C) were compared to that of the reference standard of erythromycin (FIG. 2A). These results show that peaks 1 and 2 contain isomeric erythromycins.

The results shown in FIG. 1 demonstrate that erythromycin can be chirally separated into multiple peaks. Both peaks consist of erythromycin with a molecular weight of 735 (FIG. 2B, 2C). Optical rotation for erythromycin was −76.5°, +0° for peak 1, and −59° for peak 2. The shoulder on peak 1 (FIG. 1A) and the appearance of an additional peak if an alternative mobile phase is used (see below) indicates that the chiral isolate peaks can comprise more than one optical isomer.

In an alternative embodiment, chiral separation of erythromycin was performed using a Spectra-Physics HPLC instrument and UV variable wavelength detector set at 288 nm. For chiral separation, two pre-packed 25 mm×4.6 mm ID Cyclobond I columns (Alltech Associates, 5 μm particle size) were used. The mobile phase consisted of CH₃CN: MeOH: 0.2 m ammonium acetate: H₂O (45:10:10:25 v/v) and the flow rate was 0.8 ml/min. FIG. 1B illustrates that under these conditions, at least one additional peak can be observed.

### Example 2

### Assays for Isomers of Erythromycin

Mobile Phase Preparation

The mobile phase used in this study was 56 mM sodium acetate buffer acetonitrile-methanol (56:50:4) in which the final pH was adjusted to 7.0 using concentrated acetic acid. In order to minimize the background noise the solvent mixture was pre-filtered with 0.22 μm Nylon 66 membrane filters (Fisher Scientific) and degassed using a magnetic stirrer in vacuo. The water used in the mobile phase was purified through a Milli-Q system (Millipore, Mississauga, Canada).

Sample Preparation

Frozen human plasma samples were thawed quickly (5 min) by placing the vials in warm water and aliquots (2 ml) were pipetted into 10-ml ground-glass stoppered conical extraction tubes. After the addition of internal standard (20 μl of roxithromycin solution, 750 μg/ml in acetonitrile), 5 ml of diethyl ether was added, the tubes were stoppered and then shaken vigorously for 3 min. Following centrifugation at 900 g for 5 min at 4 degrees Celsius, the upper layer was transferred into 13×100 mm disposable borosilicate tubes using a Pasteur capillary pipette and evaporated to dryness at 4 degrees Celsius under a stream of dry nitrogen (Reacti-Vap, Pierce, Rockford, Ill.). The residue was reconstituted with 100 μl of acetonitrile and vortexed for 5 s to facilitate dissolution of the sample. A 40 μl aliquot of this sample was injected onto the column. Standard curves were prepared by spiking erythromycin-free human plasma with 20 μl of concentrated acetonitrile solution of erythromycin base and estolate to yield 0, 0.5, 1, 2.5, 5, 7.5, 10 μg/1 ml or erythromycin base and ethylsuccinate to yield 0, 0.25, 0.5, 0.75, 1, 2, 3 μg/ml.

For urine samples (1.5 ml), the same preparation was used except that extraction with diethyl ether (4 ml) was preceded by the addition of 100 μl of saturated dipotassium hydrogen phosphate containing the internal standard roxithromycin at 750 μg/ml, increasing the pH from 6.5 to 8.5. Saliva samples (1.5 ml) were also extracted with diethyl ether (4 ml) and then centrifuged at 900 g for 15 min. Further steps are similar to plasma sample preparation.

In another method, the chromatographic system consisted of an HPLC pump (Perkin-Elmer LC Series 4 Pump), an autosampler (Perkin-Elmer LC-420 autosampler equipped with a 20-μl loop), a reversed-phase C₁₈ column (Whatman Partisil 5-ODS3 RAC II, 10 cm×4.6 mm I.D.), a variable-wavelength UV detector (Perkin-Elmer LC-95 spectrophotometer, set at 288 nm), and a computing integrator (Spectra-Physics SP4370). The mobile phase consisted of acetonitrile-methanol-N,N-dimethylcyclohexanamine-0.012 M dibasic ammonium phosphate (350:100:1.5:550) adjusted to pH 3.3 with acetic acid. It was filtered through a 0.45-μm Zetapor membrane, or equivalent, and degassed before use. The flow-rate was 0.5 ml/min. The preparation solvent consisted of acetonitrile-methanol-N,N diethyl-cyclohexanamine-0.012 M dibasic ammonium phosphate (350:100:1.5:550) adjusted to pH 8.8 with acetic acid. About 100 mg of erythromycin reference standard was accurately weighed and quantitatively transferred into a 100-ml volumetric flask. The standard was dissolved in and brought to volume with preparation solvent to produce a solution having a known concentration of about 1.0 mg/ml.

Ultraviolet Assay

The ultraviolet chemical assay for erythromycin remains largely unchanged from that described by Kuzel et al. (N. R. Kuzel, J. M. et al., Antibiotics and Chemotherapy, 1234-1241 (1954)). In general, the reference standard, alkali reagent, and buffer solutions are prepared prior to the assay. Phosphate buffer pH 7.0 is prepared by dissolving 13.6 g. KH₂PO₄ (anhydrous) and 27.2 g. K₂H PO₄(anhydrous) in sufficient purified water to make 5 liters. The reference standard solution is prepared by dissolving about 35 mg. accurately weighed erythromycin standard in 100 ml methanol in a 250 ml volumetric flask. This is diluted with phosphate buffer pH 7.0 to 250 ml. mixed, and allowed to cool to room temperature, then again diluted to the mark and mixed well. The alkali reagent is prepared by forming a slurry of 42 g Na₃PO₄□12H₂O in about 125 ml 0.5N NaOH in a 250 ml volumetric flask. An additional 100 ml purified water is added and the slurry heated on the steam bath to aid in solution. The solution is cooled slowly to room temperature and diluted to 250 ml with purified water, then filtered prior to use. Four 10 ml aliquots of the standard solution are pipetted into separate 25 ml volumetric flasks, two are labeled standard and the others blank. One ml of 0.5N H₂SO₄ is added to the blank flasks and they are allowed to stand after mixing at room temperature for 60 minutes±5 minutes. Two ml of purified water are added to the standard flasks. At the end of this time, 1.0 ml of 0.1 N NaOH is added to the blank flasks and their contents swirled to mix. Then, 2.0 ml of alkali reagents are added to all four flasks, they are swirled to mix and placed in a 60° C. water bath for 15.0 minutes. The flasks are then cooled rapidly in an ice bath, brought to room temperature, then diluted to 25.0 ml with purified water. The UV absorbance is read at 236 nm versus purified water in 1.0 cm silica cells. The blank values are subtracted from the standard values and the average net absorbance used for calculation.

Bulk erythromycin raw material is treated the same as the standard Formulations are made up to the same concentration as the standard is methanol and buffer and 10 ml aliquots used for chromophore development. The sulfuric acid treated aliquot representing the blank forms a cyclic ether anhydroerythromycin. The alkaline treatment causes the formation of an α,β-unsaturated ketone (9-keto-10-ene) having its absorbance maximum as a shoulder at 236 nm (E 6000). Thus, any other UV absorbing species are measured with the blank and subtracted from the absorbance before calculation of the erythromycin concentration.

Gas Chromatographic Assay

Tsuji and Robertson (Tsuji K., Robertson J H. Determination of erythromycin and its derivatives by gas-liquid chromatography. Anal Chem. 1971 June; 43 (7):818-21) reported a gas chromatographic procedure for erythromycin using an OV-225 column or a PPE-20 column. The procedure involves silylating 10 mg erythromycin with a mixture of trimethylchlorosilane, N,O-bis-trihetylsilylacetamide, and N-trimethylsilyl-imidazole in pyridine for 24 hours at 75 degrees Celsius. Ten micrograms are injected onto the column (3 mm×1850 mm, 3% OV-225 on GCQ100-120 mesh or 3 mm×1850 mm 3% PPE-20 on Supelcoport at 275° C.) of an F and M model 400 gas chromatograph equipped with a flame ionization detector. They report being able to separate erythromycins A, B, C, anhydroerythromycin A, and erythralosamine. Good agreement with the microbiological assay (see below) is shown. However, the biggest drawbacks appear to be in silylation time and the instability of the GC column, about 3 weeks at 275° C. These authors later reported using the GC method for enteric coated tablets of erythromycin, giving a recovery of 99.8% and a coefficient of variation of 2.3% based on placebo tablets spiked with erythromycin.

Colorimetric Assays

Two procedures are worthy of note. The first is based on the ion pair dye complex of bromcresol purple (5′,5″-dibromo-o-cresol-sulfonphthalein) and the desosamine moiety of erythromycin in pH 1.2 buffer (Kuzel N. R. and Coffey H. F., Technicon Symposium (1966), Vol. 2, Automation in analytical chemistry, Medical, Inc., White Plains, N.Y., 1967, pp. 235-239). This method lacks specificity for erythromycin, measuring all tertiary amines; however, it is quite sensitive and precise, being routinely used for concentrations of 250 μg/ml, in tablets and 20-100 μg/ml in fermentation broth.

Another method is also based on a complex of the desosamine moiety, but uses p-dimethyl amino benzaldehyde as the coupling agent (Sanghavi N. M. and Chandramohan H. S., Canadian Journal of Pharmaceutical Sciences 10 (2), 29-61 (1975)). This procedure is also non-specific, but sensitive and linear over a concentration range of 10-35 μg/ml.

Thin Layer Chromatography

Egon Stahl (Stahl E., Ed, Thin layer chromatography, a laboratory handbook, 2^{nd} ed., Springer-Verlag, N.Y., 1969, pp. 572) has described four TLC systems. The following Table 3 summarizes, by example, the solvents and R_{f}'s on silica gel G for erythromycin A Spraying the chromatograph with 10% molybdophosphoric acid in alcohol, followed by heating produces a blue spot on a yellow background. The spot disappears in two hours.

TABLE 3
Thin Layer Chromatography Systems

| Solvent | R_{f} | Detection |
|---|---|---|
| Methanol | 0.16 | Brownish-green color |
| | | after spraying with 10% |
| | | sulfuric acid and heating |
| | | 5-10 minutes at 80° C. |
| Chloroform:methanol | 0.03 | Brownish-green color |
| (95:5) | | after spraying with 10% |
| | | sulfuric acid and heating |
| | | 5-10 minutes at 80° C. |
| Chloroform:methanol | 0.29 | Brownish-green color |
| (50:50) | | after spraying with 10% |
| | | sulfuric acid and heating |
| | | 5-10 minutes at 80° C. |
| Butanol:acetic acid:water | 0.30 | Brownish-green color |
| (60:20:20) | | after spraying with 10% |
| | | sulfuric acid and heating |
| | | 5-10 minutes at 80° C. |

Another TLC identification system for erythromycin base, stearate, estolate and ethylsuccinate as been described that cannot differentiate between the estolate and ethylsuccinate. Other laboratories have developed a system separating erythromycin estiolate, erythromycin base, and anhydroerythromycin on a silica gel 60 F254 plate utilizing ethanol, methanol, triethylamine, 170:30:1. Visualization is by spraying with 0.15% xanthydrol and 7.5% acetic acid in water. Following Table 4 summarizes, by example, the R_{f}'s for erythromycin A.

TABLE 4

| Component | R_{f} | Color |
|---|---|---|
| Erythromycin Base | 0.30 | Violet |
| Anhydroerythromycin | 0.43 | Violet |
| Erythromycin Estolate | 0.60 | Violet |

### Example 3

### Microbiological Analysis

Kavanagh and Dennen reported microbiological tuibidimetric and plate assays for erythromycin base in Analytical Microbiology, Vol. 1. Staphylococcus aureus (ATCC 9144) is used for the turbidimetric procedure. The bulk raw material official assay is found in 21 CFR § 452.10 and the official tablet assay is found in 21 CFR § 452.110. The sample is diluted from 0.3 to 2.0 μg/ml in pH 7.0 buffer and comparison is made to a standard curve of 0, 0.3, 0.4, 0.6, 0.8, 1.2, 1.6, and 2.0 μg/ml. Sarcina lutea (ATCC 9341) is used for the plate assay. A linear response in the range of 0.5-2.0 μg/ml is obtained when pH 8.0 buffer is used for sample and standard. In both methods, a small amount of methanol is used to solubilize the erythromycin prior to buffering at pH 7.0 or 8.0.

Cylinder-Plate Method:

A cylinder-plate assay is employed to detect differences in turbidity to reflect difference in the antibiotic activity. Differences in observed turbidities reflect antibiotic activity.

To prepare assay plates using petri dishes, 21 mL of medium is placed in each of the required number of plates, and allowed to harden into a smooth base layer of uniform depth. A 4 mL of seed layer inoculum, prepared as directed for the given antibiotic, is spread the inoculum evenly over the surface and allowed it to harden. Six assay cylinders are dropped on the inoculated surface from a height of 12 mm, using a mechanical guide or other device to insure even spacing on a radius of 2.8 cm, and the plates are covered to avoid contamination. After filling the 6 cylinders on each plate with dilutions of antibiotic containing the test levels specified below, the plates are incubated at 32 to 35 degrees Celsius, or at the temperature specified for 16 to 18 hours, the cylinders are removed. The diameter of each zone of growth inhibition is measured and recorded to the nearest 0.1 mm.

For the 1-level assay with a standard curve, prepare dilutions representing 5 test levels of the standard (S₁ to S₅) and a single test level of the unknown U₃ corresponding to S₃ of the standard curve. For deriving the standard curve, fill alternate cylinders on each of 3 plates with the median test dilution (S₃) of the standard and each of the remaining 9 cylinders with one of the other four dilutions of the standard. Repeat the process for the three dilutions of the standard. For each unknown, fill alternate cylinders on each of 3 plates with the median test dilution of the standard (S₃), and the remaining 9 cylinders with the corresponding test dilution (U₃) of the unknown.

The test organism for erythromycin is Micrococcus luteus (ATCC #9341) (American Type Culture Collection, Rockville, Md.). Preparatory to an assay, remove the growth from a recently grown slant or culture of the organism, with 3 mL of sterile saline and sterile glass beads. Inoculate the surface of 250 mL of the agar medium of above and contained on the flat side of a Roux bottle. Spread the suspension evenly over the surface of the agar with the aid of sterile glass beads, and incubate at the temperature shown for approximately the indicated length of time. At the end of this period, prepare the stock suspension by collecting the surface growth in 50 mL of sterile saline, except for bleomycin (use 50 mL of medium 34).

Determine by trial the quantity of stock suspension to be used as the inoculum using methanol (10 mg/ml) final concentration 1 mg/ml. The trial tests should be incubated at 32-35° for 24 hours. The suggested inoculum composition is 1.5 m/100 ml. Adjust the quantity of inoculum on a daily basis, if necessary, to obtain the optimum dose-response relationship from the amount of growth of the test organism in the assay tubes and the length of the time of incubation. At the completion of the incubation periods tubes containing the median dose of the standard should have absorbances of at least 0.3 absorbance unit.

For the cylinder-plate assay, determine by trial the proportions of stock suspension to be incorporated in the inoculum, starting with the volumes indicated that result in satisfactory demarcation of the zones of inhibition of about 14-16 mm in diameter and giving a reproducible dose relationship. Prepare the inoculum by adding a portion of stock suspension to a sufficient amount of agar medium that has been melted and cooled to 45° to 50°, and swirling to attain a homogenous suspension.

### Example 4

### Other Analytical Methods: High Performance Liquid Chromatography

Omura used a reverse phase high performance liquid chromatographic column, JASCO PACK SV-02-500®, for macrolide antibiotics with methanol, M/15 acetate buffer pH 4.9, and acetonitrile (35:60:5) as solvent. A variable wavelength UV detector using the absorption of the individual compounds gave the required sensitivity. Alterations of buffer pH an the composition ratio of the mobile phase gave selectivity for separation of individual macrolide antibiotics.

Z. H. Hash reported chromatographic conditions for separating anhydroerythromycin from erythromycin using a normal phase Corasil II® silica gel column, with chloroform as mobile phase and refractive index detection.

White devised a reverse phase high performance liquid chromatographic procedure for erytiromycin. Refractive index detection was used since the compound absorbs weakly in the UV. A 10 μm C₁₈/Lichrosorb™ reverse phase column was used with 80% methanol, 19.9% water, 0.1% ammonium hydrochloride as the developing solvent.

Tsuji and Goetz developed a quantitative high performance liquid chromatographic method for separating and measuring erythromycins A,B and C, their epimers and degradation products. This method uses a μ Bondapak® C₁₈ reverse column with acetonitrile-methanol-0.2M ammonium acetate-water (45:10:10:25) as solvent. The pH and composition of the mobile phase may be adjusted to optimize resolution and elution volume. The authors utilized the procedure on USP reference standard and report a relative standard deviation of ±0.64%. Recently, Wardrop et al reported HPLC method for determination of erythromycins in enteric-coated tablet formulations.

Other analytical methods such as infrared spectroscopy, nuclear magnetic resonance spectroscopy, ultraviolet spectroscopy, thermal gravimetric analysis, differential thermal analysis and x-ray diffraction patterns will be used to ascertain purity and identity of these isomers.

### Example 5

### Chemical Synthesis

The erythromycins described herein can also be synthesized by total asymmetric synthesis. The overall synthetic plan was derived, with a few modifications, using the strategies and tactics of Woodward, et al. (1981) J. Am. Chem. Soc., 103: 3215-3217, and Corey, E. J., et al., (1979) J. Am. Chem. Soc., 101: 7131. Various schemes of synthesis depicted below involve site-specific operations and stereochemical control of reactions. Intermediates erythromonolides have been synthesized independently by both of these groups of workers. Following synthesis scheme, however, closely follows that of Woodward, et al. (1981).

In the preceding research report in the same volume, Woodward, et al. described the preparation of the key lactone intermediate 1a in optically active form.

These authors now report the synthesis of erythromycin (2) from 1a. In essence, this transformation involves the glycosidation of a suitable derivative of 1a with L-cladinose and D-desosamine and the generation of the C-9 ketone functionality.

Woodward, et al. were aware that glycosidation, in particular, demanded highly specific operations, in terms of both site- and stereoselectivity. The cladinose must be attached at the C-3 hydroxyl goup with α-anomeric stereochemistry and the desosamine at C-5 with β stereochemistry. The stereochemical control of these glycosidation reactions was manageable once appropriate solutions were available to the site-specific operations. The relative reactivities of the C-3 and C-5 hydroxyl groups toward glycosidation suggested a sequence of sugar attachment, as well as minimizing the need of protecting groups. 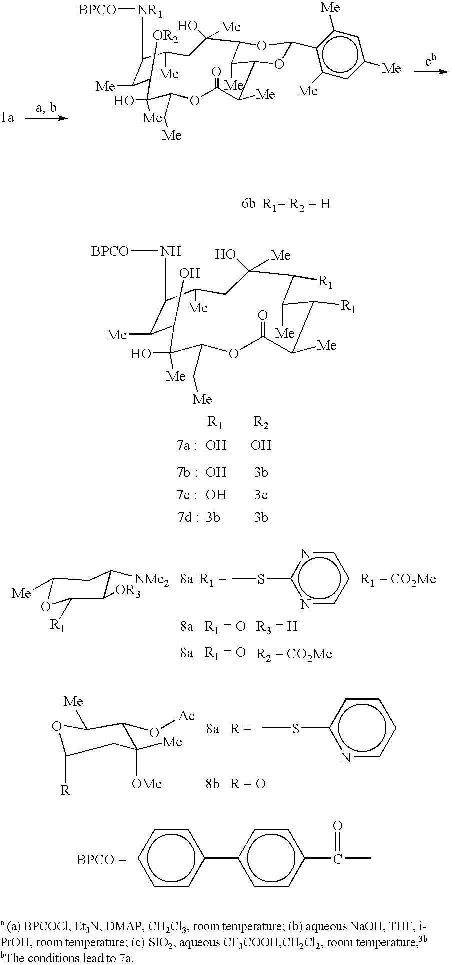
Initially the lactone 3a, derived from natural erythromycin, was chosen to study the relative reactivities of the hydroxyl groups. The attachment of L-cladinose to 3a, was studied first, since greater reactivity of the C-3 vs. the C-5 hydroxyl group was suggested by predominant formation of the 3,9,11-triacetate 3b from 3a upon acetylation (Ac₂O/Py). However, glycosidation of 3a with L-cladinal 4 (3 equiv) under modified Tatsuta conditions (3.1 equiv of N-iodosuccinimide in the presence of a radical scavenger in CH₃CN at −30° C.→25° C.) unexpectedly yielded the C5 glycoside 3c as the predominant product (34% yield based on consumed 3a; 47% conversion). The greater reactivity at C5 was further confirmed by the site-selective to attachment of D-desoamine to 3a. Thus, glycosidation of 3a using 5 (5 equiv) under modified Koenigs-Knorr conditions (10 equiv of silver triflate, lutidine, CH₂Cl₂/THF at 25° C.) yielded a single isolable glycosidation product 3d (10% yield), the desired β-glycoside at C5. These studies suggested that the C-5 hydroxyl group would be more reactive toward glycosidation, and hence protection of only the C-9 and C-11 hydroxyl groups would be sufficient.

In light of these observations, desosamine is first attached to a suitable derivative of the synthetic intermediate 1a. The 9,11-protected 1b (mp>300 degrees Celsius), readily available from 1a by CF₃COOH hydrolysis, initally appeared to be attractive, but is insoluble in almost all solvents. It therefore became necessary to first remove the cyclic carbamate (Scheme 1). By acylation with p-phenylbenzoyl chloride, carbamate 1a was converted to 6a (R1=R2=CO), hydrolysis of which produced 6b (70% yield from 1a). Deprotection of the C-3 and C-5 hydroxyl groups produced substrate 7a in quantitative yield.

Glycosidation of 7a using D-desosaminide 8a (5 equiv) and silver triflate (6 equiv) in CH₂Cl₂/PhMe at 25° C. provided the expected β-glycoside 7b [mp 172-176° C., [α]²⁵_{D}−70.7° (c 0.63,CHCl₃); 36% yield] after methanolysis. Furthermore, 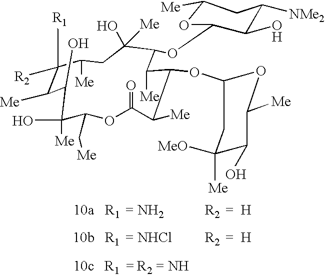
glycosidation of 7c, derived from 7b (ClCO₂Me/CH₂Cl₂/aqueous NaHCO₃), with L-cladinoside 9a (5.5 equiv) and Pb(ClO₄)₂ (6.5 equiv) in CH₃CN at 25° C., furnished after methanolysis the glycoside 7d (55% yield based on consumed 7b; 37% conversion).

Completion of the synthesis of erythromycin was carried out in the following manner. Simultaneous deprotection of both the C4″ hydroxyl group of the cladinose moiety and the C-9 amino group in 7d by Na—Hg/MeOH furnished (9S)-erythromycylamine (10a) [mp 126-129 IC, [α]²⁵D−48.1° (c 0.59,CHCl₃); 75% yield] which was found to be identical with an authentic sample prepared from natural erythromycin by a known method. Treatment of 10a with N-chlorosuccinimide (1 equiv) in pyridine at 25° C. gave 10b (mp 166-170° C. with partial melting at 130-134° C.), which was dehydrochlorinated by AgF in HMPA at 70° C. to yield erythromycinimine (10c). Hydrolysis of 10c in water at 5° C. afforded the corresponding ketone (40% overall yield from 10a), which was found to be identical with erythromycin (2) in all respects (¹H NMR, mp, mmp, α_{D}, mass, IR and chromatographic mobility. Further ability to achieve designed stereochemistry by employing stereospecfic reagents for the stereo controlled reactions can be possible.

In alternative embodiments, the isomeric erythromycins once characterized, can be converted into their corresponding esters, including, as acistrate, estolate, glucoheptonate, lactobionate, propionate, stearate, ethylsuccinate and phosphate esters. These esters act as pro-drugs thereby rendering the active erythromycins more stable before biotransformation in vivo.

Chiral separation of erythromycin isomers is performed as described above in Example 1.

### Example 6

### Effects of Erythromycin Chiral Isolates on Rat Intestine

A rat intestine preparation was used to study measure the effects of erythromycin chiral isolates on GI contractility. The ileum was harvested and placed in warmed (37° C.) Krebs Hanselest buffer and oxygenated (95% O₂ and 5% CO₂). Segments approximately 4.5 cm long were placed on a transducer (Gilson Instruments) and a preload of 2 gm was applied. The tissue was placed in a chamber and bathed in Tyrode's solution (37.5° C.) and gassed with 95% O₂ and 5% CO₂. Muscle contractions were measured isometrically. After stable contractions were obtained, a study compound was added to the tissue bath and recordings made until a new steady state of contraction was obtained. Following a washout period, contractions were recorded during a new steady state period, then the procedure repeated with at a second dose of the drug being evaluated. Changes in contraction magnitude were recorded as the percentage change from baseline. This procedure is a modification of the technique described by Van Nueten and associates (Van Nueten, et al., Inhibition of dopamine receptors in the stomach: an explanation of the gastrokinetic properties of domperidone. Life Sciences 1978, 23:453-458).

TABLE 5
The Influence of Erythromycin on GI Motility in Rat Ileum

| | Percentage change from control | | |
|---|---|---|---|
| Erythromycin | Slow Wave | Slow Wave | |
| Concentration | Amplitude | Frequency | Deflection Magnitude |
| 10⁻⁷ M | 12% | 14% | 14% |
| 10⁻⁶ M | 15% | 21% | 22% |
| 10⁻⁵ M | 17% | 27% | 33% |

n = 5 experiments

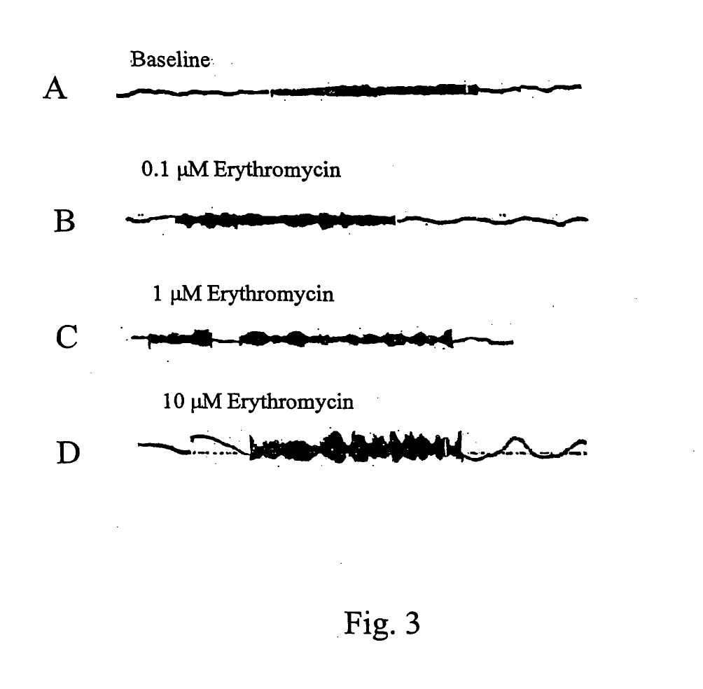

Five rat ileum segments were evaluated as to the contractile effects of erythromycin at concentrations ranging from 10⁻⁷ M to 10⁻⁵ M. As seen in Table 5, erythromycin caused a dose-related increase in slow contractile wave amplitude, slow wave frequency and the magnitude of deflection, which is proportional to contractile force. FIG. 3 shows examples of the recordings. These experiments demonstrated that erythromycin causes an increased magnitude of ileal contraction.

Using the methods described above, the effect of erythromycin and two chiral isolates on GI contractility was studied further using a rat colon preparation. A segment of rat colon was suspended in a constant temperature bath as described above, and the baseline parameters measured oxygenated Tyrode's solution without erythromycin or chiral isolates. The results are summarized in Table 6, below.

TABLE 6
The Influence of Erythromycin on GI Motility (Percent
Change in Deflection Magnitude) in Rat Colon

| | Percentage change from control | | |
|---|---|---|---|
| Concentration | Erythromycin | Peak 1 | Peak 2 |
| 10⁻⁷ M | 33% | 18% | 43% |
| 10⁻⁶ M | 70% | 33% | 75% |
| 10⁻⁵ M | 95% | 32% | 157% |

n = 5 experiments per group

### Example 6

### Measuring Potassium Channel Effects

Potassium Channel Expression and Characterization:

The two main components of the delayed rectifier potassium currents in human ventricular myocytes are I_{Kr} and I_{Ks} and are targets for class III antiarrhythmic agents. The I_{Kr} current is the product of the human ether-a-go-go related gene (HERG) and is regulated by Mink and Mirp genes. I_{Ks} is the result of co-assembly of two proteins KvLQT1 and Mink gene codified by the KVLQT1 and KCNE1 genes.

HERG encodes a K⁺ channel with biophysical properties identical to the rapid component of the cardiac delayed rectifier, I_{Kr}. It is postulated that in humans I_{Kr} is important in the re-polarization of the myocardium. The I_{Kr} current is blocked with high affinity and selectively by methasulfonamides, such as E-4031, ibutilide, and dofetilide. Inhibition of I_{Kr} causes QT prolongation. This is distinctly different from sodium inhibition, which would not cause QT interval prolongation. Inhibition of I_{Ks} would also cause an increase in action potential duration. Thus, the effects of the chiral isolates on both I_{Kr} and I_{Ks} needs to be evaluated.

The HERG pgH19 construct was a gift from Dr. Gail Robertson (University of Madison, Wis.). For cRNA injection into Xenopus oocytes the cDNA was linerarlized by NotI and in vitro transcription was made with T7 RNA polymerase using the Message Machine Kit (Ambion). cRNA expressing the HERG gene can be injecterd into Xenopus oocytes permitting the evaluation of a human channel in vitro electrophysiologic model. This offers considerable advantage in that potential problems with species differences can be avoided. Additionally, over expression of the ion channel on the cell membrane ensures that the patch clamp will record changes in voltage due to inhibition of a specific channel.

Wild type human KCNE1 and KVLQT1, cDNA was isolated from human cardiac and pancreas cDNA libraries and cloned into the pSP64 poly (A) vector (Promega). For transcription in oocytes, mink was sub-cloned from genomic DNA using the MKEL and MKER primers. The final mink expression construct contained cDNA inserted in the plasmid vector (PROMEGA). For the injection into Xenopus oocytes, complementary RNA was prepared with mCAP RNA capping Kit (Stratagene) following linearization of the expression construct by restriction digestion with EcoR1 for runoff transcription. In vitro transcription with SP6 RNA polymerase was performed using the Message Machine Kit (Ambion). The final capped-cRNA product was re-suspended in 0.1 mM KCL and stored at −80° C. The concentration of RNA synthesized was estimated by running denatured cRNA through 1.5% agarose gel together with the 0.24-9.5 Kd ladder (Gibco-BRL).

Whole cell currents were measured using a conventional two microelectrode voltage clamp technique using a Warner oocyte voltage clamp amplifier (OC-725C, Warner Instruments, Hamden, Conn.). The electrodes had impedances of between 14 megohms when filled with 3 mg/L KCl. Currents were be filtered at 1 KHz without without leak subtraction. On line data acquisition was made with a 486 IBM compatible computer and A/D converter using pCLAMP software (Axon Instruments, Foster City, Calif.). Experiments were performed at room temperature (21-23°).

KVLQT1+KCNE1 currents were recorded with a solution containing in 96 mM NaCl, 2 mM KCl, 1.8 mM CaCl₂, 10 mM MgCl₂ and 5 mM HEPES, pH 7.4. The following testing protocols were used: a) I-V plot, oocytes were held at −80 mV, then step pulses from −60 mV to +40 mV in 20 mV increments of 18 seconds were applied returning to −40 mV. b) Single pulses, the same protocol was applied for control current and after drug perfusion for current measurement. From a holding potential of −80 mV to a test potential of +40 mV during 18 seconds a pulse was applied. Then the testing component was infused continuously at a rate of 2 ml/min. over two to four minutes and the cell was either left at a resting potential of −80 mV or stimulated from a holding potential of −80 mV to a test potential of +40 mV during 1000 ms with a pacing frequency range of 0.3 Hz.

Data Analysis:

The dose response curves are fitted with a Hill equation in which IC₅₀ is the half-maximal of current inhibition and n is the Hill coefficient, and Iₒ is the amplitude of the unblocked current at the end of the test pulse. Iₘₐₓ was defined as the current amplitude of the first test pulse after drug application. Where appropriate, data from multiple measurements at each of the different drug concentrations were summarized as the mean± the standard deviation.

The currents observed have nearly identical biophysical properties to the rapid component of the cardiac delayed rectifier current, I_{Kr}. From a holding potential of −80 mV, currents are activated at potentials positive to −50 mV and have a peak current value at 0 mV. At more positive potentials, the magnitude of the current progressively decreases.

Initial experiments using chiral isolates of quinidine, a compound known to cause QT prolongation by inhibiting I_{Kr}, showed a differential effect of chiral isolates. See WO 01/46188, the teachings of which are incorporated herein by reference where not inconsistent.

Briefly, the results showed that one quinidine chiral isolate (“peak 1”) had minimal inhibitory effect on the potassium channel while another (“peak 2”) produced marked, dose-dependent inhibition of I_{Kr}. The inhibition of I_{Kr}, would lead to an increase in APD duration and thus QT prolongation. In other experiments it was also found that peak 1 of quinidine has sodium channel inhibitory activity. Peak 1 of quinidine thus has anti-arrhythmic activity, while not having the potential pro-arrhythmic action associated with QT prolongation. In other studies, the effect of different concentrations of quinidine on the I_{Ks} currents were evaluated at concentrations of quinidine from 10⁻⁶ M to 10⁻⁴ M, resulting in a 14 to 52% reduction, respectively. This inhibition of I_{Ks} is similar to that seen with amiodarone and is less than the inhibition of I_{Kr} seen across the concentrations studied. See WO 01/46188.

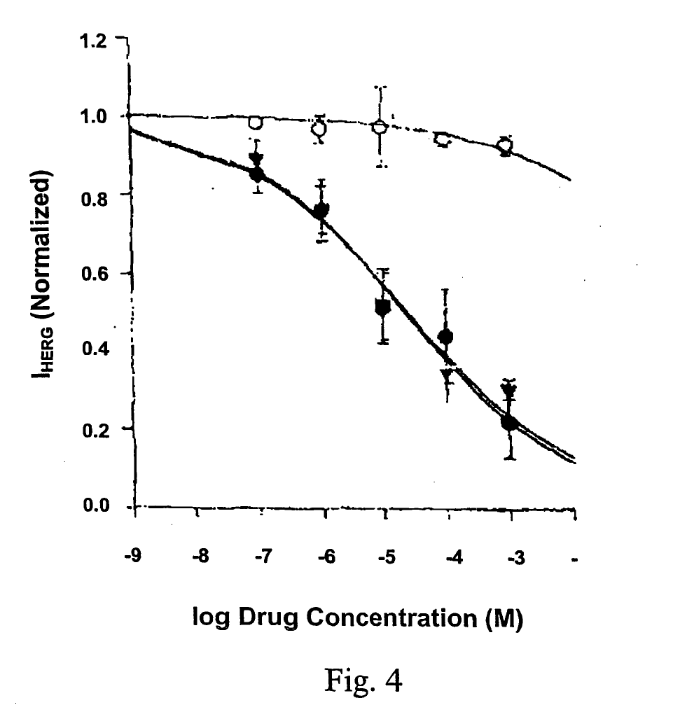

Further results shown in FIG. 4 illustrate the effect of the two erythromycin chiral isolates on the potassium channel current (I_{HERG}) measured in Xenopus oocytes, showing the effects of erythromycin chiral isolates identified in FIG. 1 as two peaks on I_{Kr}, as studied in one cell. The results show that peak 2 inhibits the potassium channel current (I_{HERC}) as does the stereoisomerically unpurified form of erythromycin, while peak 1 has essentially no effect. FIG. 4 is a graphical representation of the reduction of current through a potassium channel I_{HERG}, normalized) produced by various concentrations of erythromycin (filled triangles, IC₅₀=2.4×10⁻⁵ M), peak 1 (open circles, IC₅₀=2.3 M) and peak 2 (filled circles, IC₅₀=2.2×10⁻⁵ M). Each data point is the average of three measurements; error bars represent ± one standard deviation.

### Example 7

### Effects of Erythromycin Chiral Isolates on Gastric Secretion

Sprague-Dawley male rats (Zivic-Miller Labs, Pittsburgh, Pa., USA) weighing 70 to 90 g are allowed to acclimatize for at least 48 hours and then fasted overnight before laparotomy. Briefly, under urethane anesthesia (intra-peritoneal, 1.3 g/kg), a 15-30 cm segment of the jejunum immediately distal to the ligament of Treitz, is identified and cannulated with flexible, plastic tubing at the proximal and distal ports. The experimental solution perfusing the ileal segment is pre-warmed at 37° C. and delivered by a peristaltic pump (Harvard Instruments, Boston, Mass., USA, model 1203) at a rate of 10-12 ml/hour. The precise pumping rate is determined by measuring the rate of flow before and after the perfusion. Approximately 2 μCi/l 74 MBq/l, (NEN-Dupont, Boston, Mass., USA) of tritiated water (H₂O) is added to all solutions to determine water influx from lumen to serosa. The recirculation of the marker is estimated to be negligible during the course of the experiment. Water secretion is estimated by calculating the difference between influx and net water absorption Groups of 6 rats are perfused, and several solutions are tested in the same experiment to determine variation between experiments. After a one hour equilibration, perfusates are collected for eight 15 minute periods and analyzed for net water absorption, unidirectional fluid movement, net sodium absorption, calcium secretion into the perfusate, and glucose absorption. The rate of net water absorption is computed by the formula:
((Inflow rate(ml/min))−(Outflow rate(ml/min)/15 (min×IL(cm))×1000=μl/min×cm.

The rate of water influx, also expressed in μl/min×cm, is calculated as follows:
((H₂O dpm QRS×Inflow rate(ml/min))−H₂O dpm sample×Outflow rate (ml/min)/H₂O dpm QRS×IL(cm))×100

Electrolyte and glucose absorption are computed and expressed in nmol/mil×cm:
((Solute)QRS×Inflow rate(ml/min))−(Solute))ample×Outflow rate(ml/min)/IL(cm)×f

Where IL=intestinal length (cm); f=dilution factor×1000.

The assay results of each collection fraction are averaged and only one value is used for each experiment. Once the perfusion is ended, the rats are killed by administration of an anesthetic overdose.

The intestinal segment between the cannulae is extended with a 4 g weight and measured Tritiated water is counted in a β-scintillation counter, sodium and calcium are determined by atomic absorption spectrophotometry (Spec-trAA 10, Varian Instruments, Inc. Sunnyvale, Calif., USA) against external standards, and osmolality is measured by vapor pressure changes Model 5500, Wescor, inc., Logan, Conn., USA). Free glucose is determined enzymatically (Sigma 510). The basal perfusion medium contains sodium chloride (30 or 60 mM) and trisodium citrate (10 mM), making a total sodium concentration of either 60 or 90 mM, potassium chloride (20 mM) and glucose (111 mM=20 g/l). L-arginine (Sigma, St. Louis, Mo., USA) is added to the solutions at a 0.5, 1.0, 2.0, 10.0, or 20 mM concentration.

We claim:

1. A stereoisomerically purified form of a compound selected from the group consisting of optically active erythromycin isomeric compounds and mixtures thereof, wherein the stereoisomerically purified form has different effects on cardiac potassium ion channels and on gastric motility compared to the stereoisomerically unpurified form of the compound.

2. The stereoisomerically purified form of claim 1 wherein the stereoisomerically purified form includes from one to seven stereoisomers of erythromycin.

3. The stereoisomerically purified form of claim 1 wherein the stereoisomerically purified form has less effect on cardiac potassium channels than a stereoisomerically unpurified form of erythromycin.

4. The stereoisomerically purified form of claim 1 wherein the stereoisomerically purified form has less effect on QT prolongation than a stereoisomerically unpurified form of erythromycin.

5. The stereoisomerically purified form of claim 1 wherein the stereoisomerically purified form has less Torsade de Pointes ventricular tachycardia effect than a stereoisomerically unpurified form of erythromycin.

6. The use of the stereoisomerically purified form of claim 1 for the manufacture of a pharmaceutical composition for the treatment of infection.

7. A composition suitable for the treatment of infection comprising the stereoisomerically purified form of erythromycin of claim 1 and a pharmaceutically suitable excipient.

8. The stereoisomerically purified form of claim 1 having antibiotic efficacy similar to that of a stereoisomerically unpurified form of erythromycin, having less effect on I_{Kr} than that of a stereoisomerically unpurified form of erythromycin, and having less gastrointestinal side-effects than a stereoisomerically unpurified form of erythromycin.

9. The stereoisomerically purified form of claim 1 having antibiotic efficacy greater than that of a stereoisomerically unpurified form of erythromycin, having less effect on I_{Kr} than that of a stereoisomerically unpurified form of erythromycin, and having less gastrointestinal side-effects than a stereoisomerically unpurified form of erythromycin.

10. A method for treating infection in a patient having a need for such treatment comprising administering to the patient an effective amount of the optically active erythromycin isomeric compounds of claim 1.

11. A method for treating constipation in a patient having a need for such treatment comprising administering to the patient an effective amount of the optically active erythromycin isomeric compound of claim 1.

12. A method for treating gastroesophageal reflux disease in a patient having a need for such treatment comprising administering to the patient an effective amount of the optically active erythromycin isomeric compound of claim 1.
